(19) United States
(12) Patent Application Publication
(10) Pub. No.: US 2009/0118381 A1
(43) Pub. Date: May 7, 2009

(54) Separation of High Density Lipoproteins on Polymer Monoliths with Decreased Hydrophobicity

(75) Inventors: George Russell Warnick, Alameda, CA (US); Gary Hewett, Hayward, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Berkeley HeartLab, Inc., Burlingame, CA (US)

(21) Appl. No.: 11/950,192

(22) Filed: Dec. 4, 2007

Related U.S. Application Data
(60) Provisional application No. 60/872,666, filed on Dec. 4, 2006.

(51) Int. Cl. C07K 1/18; C08J 5/20

(52) U.S. Cl. 521/27; 530/359

(57) ABSTRACT

Described are polymer monolith compositions for separating high density lipoprotein, as well as related methods of use.

### CROSS-REFERENCE TO RELATED APPLICATIONS

This application claims the benefit of U.S. provisional patent application 60/872,666, filed Dec. 4, 2007.

### STATEMENT OF GOVERNMENT INTEREST

This invention was made with government support under grant a funded by the National Institutes of Health (R01 GM 064547-01A1) and Berkeley HeartLab, Inc. (Berlingame, Calif.). The U.S. government has certain rights in the invention.

### FIELD OF THE INVENTION

This invention is in the field of separation and purification methods for biological samples. More specifically, the invention is in the field of chromatography for the analysis, separation and purification of biological samples. In particular, in one aspect, the invention relates to poly(vinyl sulfonic acid) monolith for the separation of lipoproteins.

### BACKGROUND OF THE INVENTION

High performance liquid chromatography has grown in importance for proteomics research due to its high resolving power, excellent reproducibility and ease of interfacing with mass spectrometry (Shi et al., J. Chromatogr. A 2004, 1053, 27-36). Because of the extreme complexities of peptide mixtures in “shotgun” proteomics (Wolters et al., Anal. Chem. 2001, 73, 5683-5690), orthogonal two dimensional (2-D) liquid chromatography is required for which overall peak capacity is the product of the peak capacities of each dimension. The most widely used 2-D liquid chromatography combination is ion-exchange chromatography [especially strong cation-exchange chromatography (SCX)] followed by reversed-phase (RP) chromatography (Opiteck et al., Anal. Chem. 1997, 69, 1518-1524; Shen et al., Anal. Chem. 2004, 76, 1134-1144). For this combination, it is important to use a hydrophilic SCX column that possesses negligible mixed-mode (i.e., ion-exchange and hydrophobic interaction) retention of peptides. Otherwise, the resultant 2-D LC is not strictly orthogonal and the final overall peak capacity is compromised. In the worse case, some very hydrophobic peptides will not elute from the first dimension SCX column. Currently, the Polysulfoethyl A stationary phase, which was developed in the late 1980s (Alpert et al., J. Chromatogr. 1988, 443, 85-96; Crimmins et al., J. Chromatogr. 1988, 443, 63-71; Burke et al., J. Chromatogr. 1989, 476, 377-389), is used most widely for SCX chromatography of peptides. However, although relatively hydrophilic, the Polysulfoethyl A column has been found to exhibit some hydrophobicity, and 15-25% acetonitrile is required to suppress hydrophobic interactions to improve both peptide peak shapes and resolution (Alpert et al., J. Chromatogr. 1988, 443, 85-96; Crimmins et al., J. Chromatogr. 1988, 443, 63-71; Burke et al., J. Chromatogr. 1989, 476, 377-389).

Polymer monoliths that have comparable chromatographic performance to particle packed columns were introduced in approximately 1990 (Hjérten et al., J. Chromatogr. 1989, 473, 273-275; Svec et al., Anal. Chem. 1992, 54, 820-822.). Since their introduction, they have received considerable interest due to favorable features, such as ease of preparation and enhanced mass transfer. To date, a variety of polymer monoliths with a broad range of surface chemistries have been introduced for use in liquid chromatography. These include polymer monoliths that are based on acrylamide, methacrylate, acrylate, styrene and norbornene (Hjérten et al., J. Chromatogr. 1989, 473, 273-275; Svec et al., Anal. Chem. 1992, 54, 820-822; Palm et al., Anal. Chem. 1997, 69, 4499-4507; Ngola et al., Anal. Chem. 2001, 73, 849-856; Petro et al., J. Chromatogr. A 1996, 752, 59-66; Gusev et al., J. Chromatogr. A 1999, 855, 273-290; Premstaller et al., Anal. Chem. 2000, 72, 4386-4393; Sinner et al., Macromolecules 2000, 33, 5777-5786). In contrast to monomers used for preparation of polymer monoliths, the number of crosslinkers is much more limited. Very little effort has been directed toward study of crosslinker effects on chromatographic performance. This is quite surprising since the crosslinker is an integral part of the resulting monolith, typically accounting for 30-70% by weight. As a result, the crosslinker should be expected to significantly affect both the rigidity of the resulting monolith and its overall polarity.

Recently, we reported a new crosslinker, polyethylene glycol diacrylate (PEGDA), for the preparation of acrylate-based polymer monoliths for aqueous size exclusion chromatography of peptides and proteins (Gu et al., J. Chromatogr. A 2005, 1079, 382-391). The PEGDA crosslinker was demonstrated to have superior biocompatibility compared to conventional ethylene glycol dimethacrylate. Several other crosslinkers, including polyethylene glycol dimethacrylate (PEGDMA), were copolymerized with butyl methacrylate for reversed-phase capillary liquid chromatography of proteins (Nordborg et al., J. Sep. Sci. 2005, 28, 2401-2406). Although the advantage of the biocompatibility of the crosslinker was not demonstrated in these studies due to the use of the reversed-phase mode of chromatography, nevertheless, the feasibility of using crosslinkers other than conventional ethylene glycol dimethacrylate to prepare methacrylate-based polymer monoliths was clearly shown.

For analysis of biological samples, such as peptides and proteins, the use of PEGDA is very useful to suppress nonspecific interactions. As seen in FIG. 1, PEGDA has an acrylate group at each end of the molecule, with a PEG chain between. According to a systematic study conducted by Ostuni et al. (Langmuir 2001, 17, 5605-5620), a molecule that contains ≧3 ethylene glycol units will effectively resist the adsorption of proteins. PEG or PEG-containing materials have been widely used as slab gel matrix, capillary electrophoresis coating, capillary gel electrophoresis matrix, and artificial organ coating (Lee et al., J. Biomed. Mater Res. 1989, 23, 351-368; Tan et al., Electrophoresis, 1997, 18, 2893-2900; Zhao et al., Anal. Chem. 1993, 65, 2747-2752; Zewert et al., Electrophoresis 1992, 13, 817-824). A unique feature of PEG is that it will not denature proteins, even during precipitation at high concentration, which is in sharp contrast to other organic solvents (e.g., acetonitrile) which tend to denature proteins (Mondal et al., Anal. Chem. June 2006, 3499-3504).

With the use of PEGDA as a crosslinker, an SCX polymer monolith was recently introduced for capillary liquid chromatography of peptides (Gu et al., Anal. Chem. 2006, 3509-3518). Using simple one-step copolymerization of 2-acrylamido-2-methyl-1-propanesulfonic acid (AMPS) and PEGDA, the resulting monolith provided extremely narrow peaks and high peak capacity. Although not completely understood, the extraordinary chromatographic performance is believed to be related to the use of the biocompatible crosslinker PEGDA. In addition, it was demonstrated that excessive swelling could be avoided by using a high percentage (60 wt %) of crosslinker.

Although quite successful, an obvious drawback of the poly (AMPS-co-PEGDA) monolith is its relatively strong hydrophobicity, i.e., 40% acetonitrile is required to suppress hydrophobic interactions with hydrophobic peptides. We believe that the hydrophobicity mainly comes from the AMPS monomer because it has a 4-carbon moiety (C4) in the molecule (see the structure of AMPS, FIG. 1). In an attempt to decrease the hydrophobicity of the poly (AMPS-co-PEGDA) monolith, two other commercially available sulfonate-containing monomers, sulfoethyl methacrylate (SEMA) and vinyl sulfonic acid (VS), were investigated to prepare SCX monoliths. It was hoped that by decreasing the hydrocarbon character of the group that linked the sulfonate functionality and the acrylate or vinyl group, a monolith with decreased hydrophobicity would result. The final goal of this study was to apply the more hydrophilic monoliths to the resolution of various proteins, including lipoproteins.

### SUMMARY OF THE INVENTION

The invention provides a polymer monolith composition, comprising vinyl sulfonic acid and PEGDA. The invention further provides a polymer monolith composition, comprising sulfoethyl methacrylate and PEGDA. The invention further provides a polymer monolith composition, comprising acrylamido methanesulfonic acid and PEGDA. The invention further provides methods for separating high density lipoproteins, comprising contacting high density lipoproteins with a monolith composition according to the invention.

### BRIEF DESCRIPTION OF THE DRAWINGS

FIG. 1 shows the chemical structures of PEGDA crosslinker and several sulfonic acid-containing monomers.

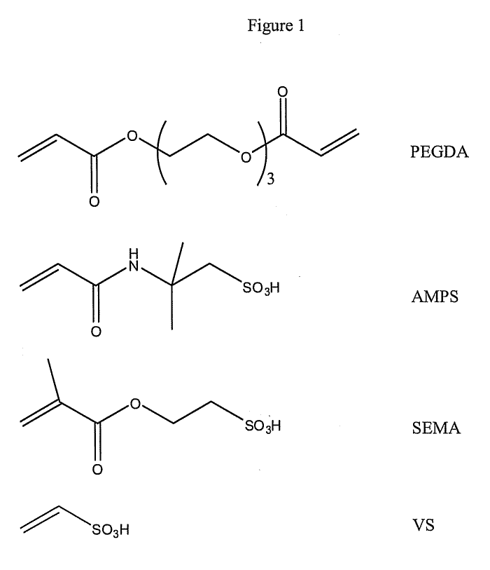

FIG. 2 shows SEM photographs of poly(SEMA) and poly(VS) monoliths. (A) poly(SEMA) monolith (scale bar=20 μm); (B) higher magnification of the monolith in (A) (scale bar=2 μm); (C) poly(VS) monolith (scale bar=20 μm); (D) higher magnification of the monolith in (C) (scale bar=2 μm).

FIG. 3 shows SCX chromatography of four synthetic undecapeptides using a poly(SEMA) monolithic column. Conditions: 15 cm×75 μm i.d. poly(SEMA) monolithic column; buffer A was 5 mM NaH₂PO₄ (pH 2.7) and buffer B was buffer A plus 1.0 (panel A) or 0.5 M NaCl (panels B, C, D and E), both buffers containing 0, 10, 20, 30, or 40% (v/v) acetonitrile (panels A, B, C, D, and E, respectively); 2 min isocratic elution of 1% B, followed by a linear AB gradient (5% B/min for panels B, C, D and E, and 2.5% B/min for panel A) to 100% B and various times of isocratic elution with 100% B until peptide 4 was eluted; 1.8 min gradient delay time; mixture of peptides 1-4 (c.f. Table 1 in Gu et al., Anal. Chem. 2006, 3509-3518); 12 μL/min pump master flow rate; 510, 460, 440, 440 or 440 mL/min column flow rates (panels A, B, C, D, and E, respectively); online UV detection at 214 nm.

FIG. 4 shows fast SCX chromatography of synthetic peptides using a poly(SEMA) monolithic column. Conditions were the same as in FIG. 3E except that a faster pump master flow rate of 48 μL/min, column flow rate of 1.9 μL/min and 20% B/min gradient rate were used.

FIG. 5 shows SCX chromatography of synthetic peptides using a poly(VS) monolithic column. Conditions were the same as in FIG. 3 with the following exceptions: 16 cm×75 μm i.d. poly(VS) monolithic column; Buffer B in panel A contained 0.5 M NaCl; pump master flow rate was 24 μL/min; gradient delay time was 8 min; 102, 98, 83, 83 or 78 mL/min column flow rates (panels A, B, C, D, and E, respectively).

FIG. 6 shows SCX chromatography of proteins using a poly(VS) monolithic column. Conditions: 16 cm×75 μm i.d. poly(VS) monolithic column; buffer A was 5 mM phosphate (pH 6.2) and buffer B was buffer A plus 1.0 M NaCl; 24 μL/min pump master flow rate; column flow rate was 104 mL/min; gradient delay time was 8 min; linear gradient from 1% B to 50% B in 20 min, ramped to 100% B in 2 min and held for 20 min; analytes: (1) 1.14 mg/mL of cytochrome c, (2) 1.60 mg/mL of α-chymotrypsinogen A, (3) 1.10 mg/mL of ribonuclease A and (4) 1.50 mg/mL of lysozyme; the baseline drift during gradient elution and the rise of the baseline at the end of the gradient were due to the difference in UV absorbances of buffers A and B.

FIG. 7 shows SCX chromatography of high density lipoprotein using a poly(VS) monolithic column. Conditions were the same as in FIG. 6 with the following exceptions: buffer A was 10 mM citrate (pH 5.0) containing 0.01% EDTA, and buffer B was buffer A plus 1.0 M NaCl; 2 min 1% B, 20 min gradient from 1% to 100% B, and 12 min 100% B; 11 mg/mL HDL; online UV detection at 214 nm.

### DETAILED DESCRIPTION OF THE INVENTION

Two polymer monoliths were designed and synthesized from commercially available monomers with an attempt to decrease hydrophobicity for strong cation-exchange chromatography. One was prepared from the copolymerization of sulfoethyl methacrylate and polyethylene glycol diacrylate, and the other was synthesized from vinyl sulfonic acid and polyethylene glycol diacrylate. Both of the monoliths were synthesized inside 75 μm i.d. UV transparent fused silica capillaries by photopolymerization. The hydrophobicities of the two monoliths were systematically evaluated using standard synthetic undecapeptides. The poly(sulfoethyl methacrylate) monolith demonstrated similar hydrophobicity as a monolith prepared from copolymerization of 2-acrylamido-2-methyl-1-propanesulfonic acid and polyethylene glycol diacrylate, and 40% acetonitrile was required to suppress any hydrophobic interactions with solutes. However, with the use of vinyl sulfonic acid as the functional monomer, a monolith with very low hydrophobicity was obtained, making it suitable for strong cation-exchange liquid chromatography of both peptides and proteins. It was found that monolith hydrophobicity could be adjusted by selection of monomers that differ in hydrocarbon content and type of vinyl group. Finally, excellent separations of model protein standards and high density lipoproteins were achieved using the poly(vinyl sulfonic acid) monolith. Five subclasses of high density lipoproteins were resolved using a simple linear NaCl gradient.

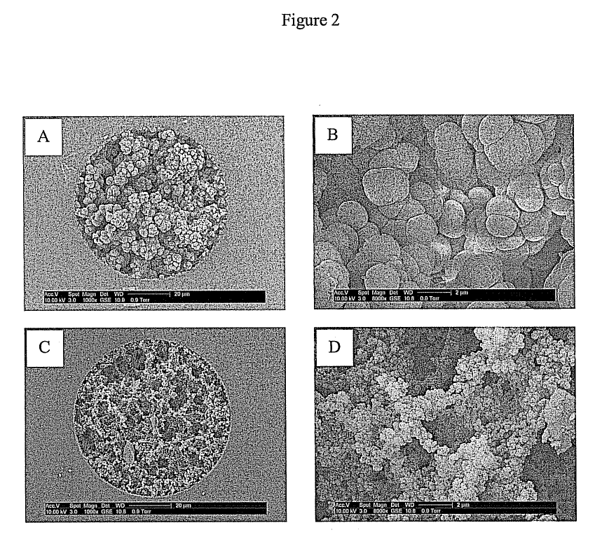

Preparation of Polymer Monoliths. The proper selection of porogen is of paramount importance in the preparation of a monolith for use in chromatography. Because PEGDA was used as crosslinker and sulfonate-containing monomers similar to AMPS were used, the initial choice of porogen was a mixture of water, methanol and ethyl ether as used previously (Gu et al., Anal. Chem. 2006, 3509-3518). Although polymer monoliths were formed using this recipe, cracks along the axis of the capillary were observed under an optical microscope. This resulted in monoliths with extremely low flow resistance and poor column efficiency, because of channeling of the mobile phase through the cracks in the monolith. New porogens had to be found to prepare the poly(SEMA) monolith. After extensive screening, a binary porogen composed of ethyl ether and hexanes yielded monoliths that were macroscopically uniform and possessed very low flow resistance. Further optimization, however, revealed that hexanes were unnecessary to be included as a coporogen. The optimized reagent composition for the poly(SEMA) monolith is given in Table 1. FIG. 2 shows an SEM photograph of the poly(SEMA) monolith. The monolith was attached to the capillary wall, and no cracks were observed. A morphology typical to conventional polymethacrylate monoliths was obtained.

TABLE 1
Reagents and dynamic binding capacities of poly(AMPS), poly(SEMA) and poly(VS) monoliths.

| | Reagent | | | | | | | Dynamic binding capacity^{a} | |
|---|---|---|---|---|---|---|---|---|---|
| | DMPA | Monomer | PEGDA | Water | Methanol | Ethyl ether | UV time | Peptide | Protein |
| | (g) | (g) | (g) | (g) | (g) | (g) | (min) | (μequiv/mL) | (mg/mL) |
| AMPS^{b} | 0.008 | 0.32 | 0.48 | 0.20 | 0.55 | 1.70 | 3 | 157 | 332 |
| SEMA | 0.008 | 0.32 | 0.48 | / | / | 0.80 | 30 | 62 | 8 |
| VS | 0.0008 | 1.07^{c} | 0.48 | / | 0.75 | / | 3 | 11 | 32 |

^{a}Dynamic binding capacity was measured based on the uptake of bradykinin fragment 1-7 (peptide) or cytochrome c (protein). For experimental conditions, please refer to ref 24.
^{b}The reagents and dynamic binding capacity for poly(AMPS) monolith are from ref 24.
^{c}The VS monomer is a 30 wt % water solution.

The preparation of the poly(VS) monolith was originally thought to be somewhat challenging because the VS monomer could only be obtained as a 30 wt % water solution and not in the neat form. This introduced the requirement that water must be included in the monolith recipe and the weight ratio between VS and water had to be equal to (no addition of water) or less than 3/7 (with addition of water). Fortunately, the preparation of the poly(VS) monolith was far less difficult than anticipated. A combination of water and methanol was found effective in generating a stable flow-through monolith. The optimized reagent composition is listed in Table 1. SEM of the optimized poly(VS) monolith (FIG. 2) revealed a different morphology compared to the poly(SEMA) monolith, but a similar morphology to the poly(AMPS) monolith.

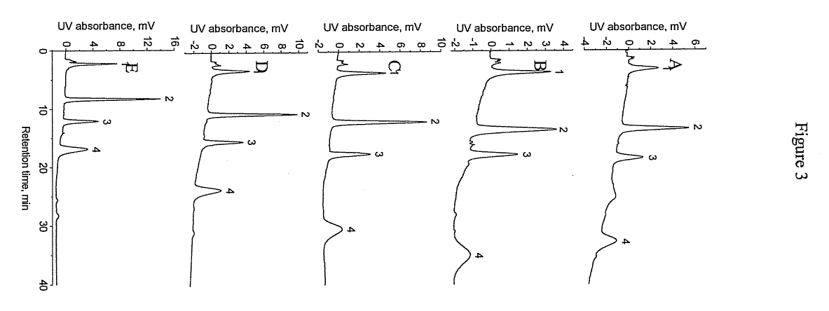

Hydrophobicity of the Poly(SEMA) Monolith. Four synthetic undecapeptides (see Table 1 in Gu et al., Anal. Chem. 2006, 3509-3518) were used to determine the hydrophobicity of the poly(SEMA) and poly(VS) monoliths. FIG. 3 shows a gradient elution separation of the four synthetic peptides using buffers that contain different amounts of acetonitrile. For the most hydrophobic peptide 4 with hydrophobicity index 24.2 at pH 2.0 (Gu et al., Anal. Chem. 2006, 3509-3518), 40% acetonitrile was required to suppress the hydrophobic interaction between the monolith and the peptide. For the other three peptides, there was negligible difference between the elution patterns between 0% to 20% acetonitrile additives. However, when higher concentrations of acetonitrile (e.g., 30 or 40%) were used in the mobile phase, narrower peaks were observed. In general, the elution pattern of the four synthetic peptides using poly(SEMA) were similar to that of the poly(AMPS) monolith. However, much lower column efficiency was observed for the newly prepared poly(SEMA) monolith, although resolution of the four peptides was acceptable. A peak capacity of 21 was achieved for the poly(SEMA) column using buffers containing 40% acetonitrile, in contrast to 71 for the poly(AMPS) monolith.

It is surprising that the hydrophobicity of poly(SEMA) is similar to that of poly(AMPS) although there is less hydrocarbon character in the SEMA molecule. Therefore, for overall hydrophobic interaction, other factors must be considered. Due to the single bond connection to the monolith backbone, the sulfonate functional group would rotate freely into and out of the backbone. In some circumstances, analytes could directly interact with the backbone of the monolith. Although the contribution to hydrophobicity by the biocompatible PEGDA crosslinker was found to be insignificant, the carbon-carbon linkage resulting from polymerization of vinyl groups in the monomer could lead to some hydrophobic interactions. Thus, the overall hydrophobicity must result from the sum of the hydrocarbon components of the side chains of the functional groups and the backbone of the polymer.

The backbone hydrophobicity is mainly determined by the type of vinyl group and the surface coverage by the functional groups. At present, there is no good methodology available to directly measure the surface coverage by the functional groups. One indirect method is to use dynamic binding capacity to estimate the surface coverage. The dynamic binding capacity of the poly(SEMA) monolith was measured to be 62 μequiv/mL, based on the uptake of bradykinin fragment 1-7 (see Table 1). This value is smaller than that of the poly(AMPS) monolith (157 μequiv/mL), indicating a lower surface coverage by the sulfonate groups; this results in less hydrophobicity. However, another more important factor that affects the backbone hydrophobicity is the type of vinyl groups in the monomer. The backbone hydrophobicity of poly(AMPS) is low because of the use of the biocompatible acrylamido group. As a result, the overall hydrophobicity of poly(SEMA) (from the C2 sulfonate linkage and the backbone) is comparable to that of poly(AMPS) (mainly from the C4 linkage).

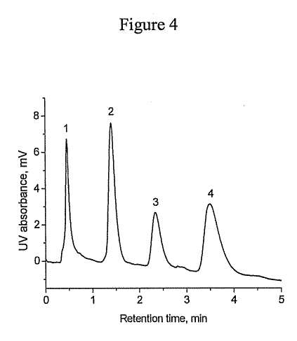

Although disappointing for decreasing column hydrophobicity, the poly(SEMA) monolith had very low flow resistance, which made it useful in performing fast separations. FIG. 4 shows a separation of the four undecapeptide standards in 5 min using a fast flow rate (linear velocity of 43 cm/min) and a sharp gradient. The total analysis time could be further decreased to 2 min by simply using a sharper gradient rate (50% B/min). However, the peak for peptide 4 became somewhat skewed under these conditions.

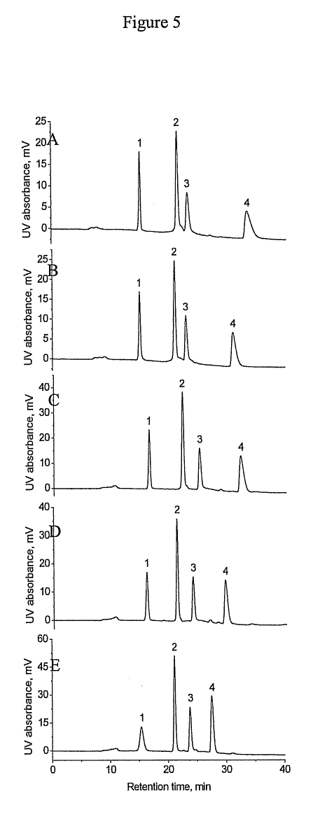

Hydrophobicity of the Poly(VS) Monolith. FIG. 5 shows the elution of the four synthetic peptides under various acetonitrile concentrations. It is obvious that the overall hydrophobicity of the poly(VS) column is much less than either poly(AMPS) or poly(SEMA) monoliths. As is seen in FIG. 5a for which no acetonitrile was used, peptide 4 could be eluted in 40 min, although a tailing peak was observed due to nonspecific hydrophobic adsorption. With an acetonitrile concentration of 30%, hydrophobic interactions could be suppressed, and 40% acetonitrile narrowed the peptide 4 peak somewhat further. The resolution of peptides 2 and 3 was improved with the addition of 20% acetonitrile. Although improvement was made with the addition of acetonitrile, the effect of acetonitrile on the peak profiles for peptides 2 and 3 was not as dramatic as for either poly(AMPS) or poly(SEMA) monoliths, indicating decreased hydrophobicity of the poly(VS) monolith.

Peak capacity was increased from 20 (FIG. 5a) to 27 (FIG. 5d) with the addition of 30% acetonitrile compared to no acetonitrile, and decreased to 24 (FIG. 5e) when 40% acetonitrile was used. Therefore, 20-30% acetonitrile is sufficient to suppress the hydrophobic interaction of the poly(VS) monolith. The hydrophobicity of the poly(VS) monolith must come from the backbone of the monolith because the VS monomer does not have any extra carbon atoms in the linking group. While the dynamic binding capacity of the poly(VS) monolith was smaller than that of the poly(AMPS) monolith, indicating less hydrophobicity, a significant contribution to column hydrophobicity could still come from the backbone of the monolith. Although somewhat hydrophobic, it should be noted that the poly(VS) monolith could elute the most hydrophobic peptide 4 in relatively short time without the addition of acetonitrile, making it useful as a first dimension in proteomics studies.

The column stability and reproducibility of the poly(VS) monolith are excellent. The poly(VS) monolith was continuously used at ˜1000 psi head pressure for two months without deterioration of column performance (i.e., resolution, efficiency and peak shape). This confirms that it is feasible to prepare a stable SCX monolith by copolymerization of a sulfonate-containing monomer and a crosslinker if high percentage of crosslinker is used. An evaluation of run-to-run reproducibility with buffers containing 30% acetonitrile, gave relative standard deviation values (RSD, n=5) of retention times and peak heights for the four synthetic peptides of 1.5, 1.0, 0.8, and 0.5, and 2.5, 1.2, 2.0, and 1.6, respectively. Column-to-column reproducibility was also good; the RSDs (n=3) for retention times and peak heights were 2.5, 1.4, 1.6, and 3.0, and 2.3, 2.8, 1.6, and 4.0, respectively.

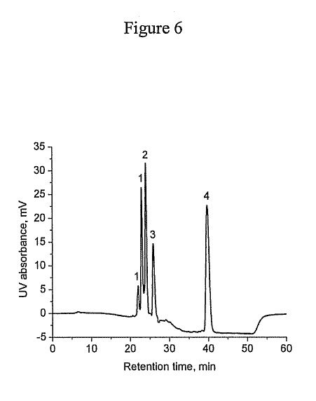

Strong Cation-Exchange Liquid Chromatography of Proteins. FIG. 6 shows SCX chromatography of protein standards using the hydrophilic poly(VS) monolith. Sharp peaks were obtained for all four proteins. Although the poly(VS) monolith generated lower peak capacity for the four undecapeptides than did the poly(AMPS) monolith, it yielded better peak profiles for proteins. This indicates that a polymer monolith with less hydrophobicity was prepared. It also demonstrates that a monolith with carefully designed hydrophilicity is beneficial for SCX chromatography of proteins.

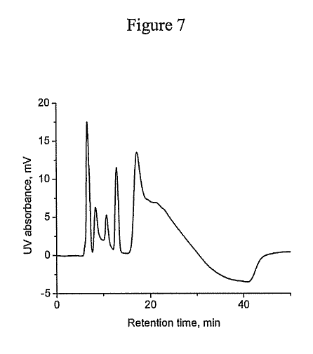

The usefulness of the poly(VS) monolith was further demonstrated by SCX chromatography of hydrophobic proteins. Lipoproteins are important biological macromolecular complexes of lipids and apolipoproteins which function to transport lipids in blood (Otvos et al., Handbook of Lipoprotein Testing, 2nd ed.; AACC Press: Washington D.C., 2000). Disorders in lipoprotein metabolism is one of the most important risk factors for the development of coronary heart disease. Because they contain lipids and are bulky, lipoproteins are very hydrophobic and, thus, difficult to analyze using conventional SCX columns (Hirowatari et al., Anal. Biochem. 2002, 308, 336-342). High density lipoprotein (HDL) is a very complex mixture that has been resolved into 12 subclasses using 2-D gel eletrophoresis (Asztalos et al., Biochim. Biophys. Acta, 1993, 1169, 291-300). Using the hydrophilic poly(VS) monolith, five subclasses of HDL were resolved (FIG. 7). Further optimization of chromatographic parameters for this application is underway.

Conclusions. In this study, we prepared stable SCX monoliths by copolymerizing sulfonate-containing monomers and PEGDA crosslinker. In the design of SCX polymer monoliths for peptides and proteins, it is important to control the overall hydrophobicity to decrease nonspecific interactions. The overall hydrophobicity of the monolith can be tuned by the use of appropriate crosslinkers and monomers. PEGDA greatly decreased monolith backbone hydrophobicity compared to ethylene glycol dimethacrylate. The contribution of hydrophobicity from the monomer mainly results from the linking group that connects the sulfonate functionality with the polymerization functionality. The type of polymerization functionality (e.g, vinyl or methacrylate or acrylamido) also results in different backbone hydrophobicity. Among the three monomers (AMPS, SEMA and VS) studied, VS resulted in a monolith with the least hydrophobicity.

Further improvement should be achieved with the use of more suitable monomers. For example, if acrylamido methanesulfonic acid is used as a functional monomer and PEGDA as a crosslinker, an SCX monolith with negligible hydrophobicity would be expected. Although we have already synthesized this monomer, we have not been able to purify it sufficiently. Another potentially useful monomer would have acrylate or methacrylate at one end, PEG in the middle, and sulfonate at the other end. By using PEGDA as a crosslinker, an ideal monolith with backbone completely comprised of PEG and surface comprised of sulfonate would be obtained.

The invention is further described in the following non-limiting examples.

### EXAMPLES

Safety Considerations. The SEMA and VS monomers, and the PEGDA crosslinker are sensitizing agents. Appropriate MSDS information should be consulted for handling of these materials. Sunglasses that block UV light and gloves should be worn to avoid burns caused by the high-power UV-curing system during the preparation of the monoliths.

### Example 1

Chemicals and Reagents. 2,2-Dimethoxy-2-phenyl-acetophenone (DMPA, 99%), 3-(trimethoxysilyl)propyl methacrylate (98%), and poly(ethylene glycol) diacrylate (PEGDA, Mn˜258) were purchased from Sigma-Aldrich (Milwaukee, Wis.) and used as received. Sulfoethyl methacrylate was obtained from Polysciences (Warrington, Pa.), and vinyl sulfonic acid sodium salt (30% aqueous solution) was purchased from Sigma-Aldrich. Both of the monomers were used without further purification. Porogenic solvents for monolith synthesis and chemicals for mobile phase buffer preparation were HPLC or analytical reagent grade.

Bradykinin fragment 1-7 and proteins (myoglobin from equine skeletal muscle, cytochrome c from bovine heart, α-chymotrypsinogen A from bovine pancreas and lysozyme from chicken egg white) were obtained from Sigma-Aldrich. Synthetic peptide standard CES-P0050 was purchased from Alberta Peptides Institute (Edmonton, Alberta, Canada). High density lipoprotein (HDL) was from Calbiochem (La Jolla, Calif.). Ethylenediaminetetraacetic acid (EDTA, disodium salt, dehydrate, ultrapure grade) was provided by Invitrogen (Carlsbad, Calif.).

### Example 2

Polymer Monolith Preparation. UV transparent fused silica capillary tubing (75 μm i.d., 375 μm o.d., Polymicro Technologies, Phoenix, Ariz.) was silanized with 3-(trimethoxysilyl)propyl methacrylate to provide a pendant vinyl group for anchoring of polymer monoliths following a procedure developed by Vidi\check{c} et al. with slight modifications (Vidi\check{c} et al., J. Chromatogr. A 2005, 1065, 51-58). Briefly, a 5 m long capillary was rinsed sequentially with ethanol and water. The capillary was then filled with 2 M HCl, and heated at 110° C. for 3 h in a GC oven with both ends sealed with a union (Upchurch, Oak Harbor, Wash.). After surface activation, the capillary was rinsed again with water and ethanol, and dried at 120° C. for 1 h under a nitrogen gas purge. Silanization of the surface activated capillary was performed with 15% (v/v) TPM in dry toluene at 35° C. overnight. After silanization, the capillary was washed with toluene and acetone sequentially, and then dried under a nitrogen gas purge at room temperature overnight. Both ends of the silanized capillary were sealed with rubber septa until further use.

Monomer solutions (see Table 1 for reagent compositions) were prepared in 1-dram (4 mL) glass vials by admixing initiator, monomer, crosslinker and porogens. The monomer solutions were ultrasonicated for 10 s, introduced into the surface silanized capillary by capillary action, and irradiated for a certain amount of time using a UV curing system reported elsewhere. After the monoliths were prepared, they were connected to an HPLC pump and flushed with methanol and water to remove porogens and any unreacted monomers. Scanning electron micrographs (SEM) of the monoliths were obtained as previously described (Gu et al., Anal. Chem. 2006, 3509-3518).

### Example 3

Capillary Liquid Chromatography (CLC). The CLC system used in this study was described in detail elsewhere (Gu et al., Anal. Chem. 2006, 3509-3518). To decrease the system delay time and set the split ratio to ˜1:1000, the splitter capillary was changed to 40 cm long×30 μm i.d., and the original stainless steel tubing (100 cm long× 1/32 inch o.d.×200 μm i.d.) from the mobile phase mixer was replaced with an open tubular capillary (70 cm long×360 μm o.d.×75 μm i.d.). The chromatographic conditions are given in the figure captions. The dynamic binding capacities of the test peptides and proteins were measured, following exactly the procedure previously described (Gu et al., Anal. Chem. 2006, 3509-3518).

We claim:

1. A polymer monolith composition, comprising copolymerized vinyl sulfonic acid and PEGDA.

2. The composition of claim 1, further comprising DMPA and methanol.

3. A method for separating high density lipoprotein, comprising contacting high density lipoprotein with a monolith composition according to claim 1.

4. A method for separating high density lipoprotein, comprising contacting high density lipoprotein with a monolith composition according to claim 2.

5. A polymer monolith composition, comprising copolymerized sulfoethyl methacrylate and PEGDA.

6. The composition of claim 5, further comprising DMPA and ethyl ether.

7. A method for separating high density lipoprotein, comprising contacting high density lipoprotein with a monolith composition according to claim 5.

8. A method for separating high density lipoprotein, comprising contacting high density lipoprotein with a monolith composition according to claim 6.

9. A polymer monolith composition, comprising copolymerized acrylamido methanesulfonic acid and PEGDA.

10. A method for separating high density lipoprotein, comprising contacting high density lipoprotein with a monolith composition according to claim 9.
